(19) United States
(12) Patent Application Publication
(10) Pub. No.: US 2026/0188011 A1
(43) Pub. Date: Jul. 2, 2026

(54) VIDEO ANALYSIS DASHBOARD FOR CASE REVIEW

(71) Applicant: DIGITAL SURGERY LIMITED, London (GB)

(72) Inventors: Carole RJ Addis, London (GB); Imanol Luengo Muntion, London (GB); Karen Kerr, London (GB); Danail V. Stoyanov, London (GB); Stefano Passaretti, London (GB)

(21) Appl. No.: 19/131,380

(22) PCT Filed: Nov. 22, 2023
(86) PCT No.: PCT/EP2023/082732

§ 371 Date: May 20, 2025

Related U.S. Application Data
(60) Provisional application No. 63/427,222, filed on Nov. 22, 2022.
(60) Provisional application No. 63/510,525, filed on Jun. 27, 2023.

(51) Int. Cl. G06V 20/40; G06V 20/50; G16H 50/70

(52) U.S. Cl. CPC G06V 20/47; G06V 20/50; G16H 50/70

(57) ABSTRACT

Examples described herein provide a computer-implemented method that includes receiving a video of a surgical procedure. The method further includes analyzing the video of the surgical procedure to identify a feature of the surgical procedure. The method further includes generating a video analysis dashboard based at least in part on the feature of the surgical procedure. The video analysis dashboard includes a video region to display the video of the surgical procedure, a case summary region to display a case summary of the surgical procedure, a timeline reel region to display timelines of the surgical procedure, which can be used to jump to selected parts of the video and to create highlight reels, and an analytics region to display analytics of the surgical procedure.

### BACKGROUND

The present invention relates in general to computing technology and relates more particularly to computing technology for a video analysis dashboard for case review.

Computer-assisted systems, particularly computer-assisted surgery systems (CASs), rely on video data digitally captured during a surgery. Such video data can be stored and/or streamed. In some cases, the video data can be used to augment a person's physical sensing, perception, and reaction capabilities. For example, such systems can effectively provide the information corresponding to an expanded field of vision, both temporal and spatial, that enables a person to adjust current and future actions based on the part of an environment not included in his or her physical field of view. Alternatively, or in addition, the video data can be stored and/or transmitted for several purposes, such as archival, training, post-surgery analysis, and/or patient consultation. The process of analyzing and comparing a large amount of video data from multiple surgical procedures to identify commonalities can be highly subjective and error-prone due, for example, to the volume of data and the numerous factors (e.g., patient condition, physician preferences, and/or the like including combinations and/or multiples thereof) that impact the workflow of each individual surgical procedure that is being analyzed.

### SUMMARY

In one exemplary embodiment, a computer-implemented method is provided. The method includes receiving a video of a surgical procedure. The method further includes analyzing the video of the surgical procedure to identify a feature of the surgical procedure. The method further includes generating a video analysis dashboard based at least in part on the feature of the surgical procedure. The video analysis dashboard includes a video region to display the video of the surgical procedure, a case summary region to display a case summary of the surgical procedure, a timeline reel region to display timelines of the surgical procedure, which can be used to jump to selected parts of the video and to create highlight reels of the surgical procedure, and an analytics region to display analytics of the surgical procedure.

In addition to one or more features described herein, a system is described, including a processor configured to receive a video of a surgical procedure from non-transitory memory, wherein the processor is configured to analyze the video of the surgical procedure to identify a feature of the surgical procedure and generate a video analysis dashboard based at least in part on the feature of the surgical procedure, the video analysis dashboard comprising: a video region to display the video of the surgical procedure; a case summary region to display a case summary of the surgical procedure; a a timeline reel region to display timelines of the surgical procedure, which can be used to jump to selected parts of the video and to create highlight reels of the surgical procedure; and an analytics region to display analytics of the surgical procedure; and wherein a display provides a visual display of the output from the video analysis dashboard.

In addition to one or more of the features described herein, or as an alternative, further embodiments of the method or system may include that a timeline reel region to display timelines of the surgical procedure, which can be used to jump to selected parts of the video and to create highlight reels and download the highlight reel.

In addition to one or more of the features described herein, or as an alternative, further embodiments of the method or system may include that the video includes an augmented reality element associated with a feature of the video.

In addition to one or more of the features described herein, or as an alternative, further embodiments of the method or system may include that the case summary comprises a plurality of key moments and timestamps associated with each of the plurality of key moments.

In addition to one or more of the features described herein, or as an alternative, further embodiments of the method or system may include that the key moments are identified using a machine learning algorithm or a statistical analysis.

In addition to one or more of the features described herein, or as an alternative, further embodiments of the method or system may include that the timeline reel region comprises at least one selected from the group consisting of an events timeline, a phases timeline, a camera timeline, a surgeons timeline, one or more anatomy timelines, and one or more instruments timelines.

In addition to one or more of the features described herein, or as an alternative, further embodiments of the method or system may include that the timeline reel region comprises an add anatomy option to add an anatomy to the timeline.

In addition to one or more of the features described herein, or as an alternative, further embodiments of the method or system may include that the timeline reel region comprises an add instruments option to add an instrument to the timeline.

In addition to one or more of the features described herein, or as an alternative, further embodiments of the method or system may include that the analytics region comprises at least one selected from the group consisting of a metrics overview, a phase analysis, an energy instrument usage, a critical structure viability, user created metrics, and user or pre-defined procedure specific metrics.

In addition to one or more of the features described herein, or as an alternative, further embodiments of the method or system may include that data for the analytics region is based on an average in general or for cases with the same case tags, wherein the average is an average for a particular surgeon who performed the surgical procedure, an average for a group of surgeons associated with the particular surgeon, or a global average for a global population of surgeons.

In addition to one or more of the features described herein, or as an alternative, further embodiments of the method or system may include that data for the analytics region is based on a statistical analysis of the video, wherein the statistical analysis includes determining an average, a standard deviation and outlier detection.

In addition to one or more of the features described herein, or as an alternative, further embodiments of the method or system may include that the video analysis dashboard further comprises a similar case region to display oner or more similar cases relative to the surgical procedure.

In addition to one or more of the features described herein, or as an alternative, further embodiments of the method or system may include that the video is captured by a camera.

In addition to one or more of the features described herein, or as an alternative, further embodiments of the method or system may include that the video is captured by an ultrasound device.

The above features and advantages, and other features and advantages, of the disclosure are readily apparent from the following detailed description when taken in connection with the accompanying drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 depicts a computer-assisted surgery (CAS) system according to one or more embodiments described herein;

FIG. 2 depicts a surgical procedure system according to one or more embodiments described herein;

FIG. 3 depicts a system for analyzing video captured by a video recording system according to one or more embodiments described herein;

FIG. 4 depicts a flow diagram of a method according to one or more embodiments described herein;

FIG. 5A-5T depict an example of a video analysis dashboard according to one or more embodiments described herein; and

FIG. 6 depicts a block diagram of a computer system according to one or more embodiments described herein.

The diagrams depicted herein are illustrative. There can be many variations to the diagrams and/or the operations described herein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Exemplary aspects of the technical solutions described herein include systems and methods for a video analysis dashboard for case review. As described herein, “video” can refer to video captured by an imaging device (e.g., a camera), images captured by an ultrasound device, or other imaging modalities.

In some situations, it may be desirable to review a surgical procedure after the surgical procedure has been complete. Such post-surgical procedure review provides interested parties, such as surgeons, assistants, nurses, administrators, and/or any other actors, with the ability to review the surgical procedure.

Contemporary approaches to post-surgical procedure review do not provide comprehensive information about the surgical procedure or a view for quickly and easily consuming such information. In particular, contemporary approaches do not provide for interested parties to review inefficiencies, variations, and key surgical events.

According to an embodiment, a video analysis dashboard is provided that shows a video of a surgical procedure and an analysis for the surgical procedure using data from machine learning algorithms and manual annotation to provide for post-surgical procedure review. For example, the video analysis dashboard provides analytics based on general case overview, surgical workflow, anatomy, critical structures, surgical instruments, events, and/or the like, including combinations and/or multiples thereof. According to one or more embodiments described herein, the video analysis dashboard presents insights extracted from surgical video using machine learning algorithms and manual annotation to enable interested parties to review information about the surgical procedure, such as inefficiencies, variations, and key surgical events. These insights have not previously been available to those interested parties in contemporary approaches to post-surgical procedure review.

One or more embodiments of a video analysis dashboard as described herein combine visual representations of analytics for instrument, anatomy, multiple surgeon, and surgical event annotations where contemporary approaches to post-surgical procedure review provide only phases and events.

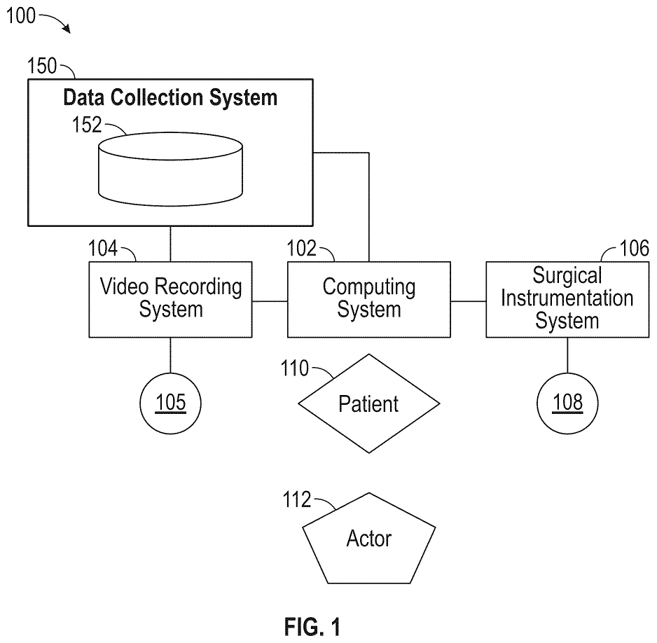

Turning now to FIG. 1, an example computer-assisted system (CAS) system 100 is generally shown in accordance with one or more aspects. The CAS system 100 includes at least a computing system 102, a video recording system 104, and a surgical instrumentation system 106. As illustrated in FIG. 1, an actor 112 can be medical personnel that uses the CAS system 100 to perform a surgical procedure on a patient 110. Medical personnel can be a surgeon, assistant, nurse, administrator, or any other actor that interacts with the CAS system 100 in a surgical environment. The surgical procedure can be any type of surgery, such as but not limited to cataract surgery, laparoscopic cholecystectomy, endoscopic endonasal transsphenoidal approach (eTSA) to resection of pituitary adenomas, or any other surgical procedure. In other examples, actor 112 can be a technician, an administrator, an engineer, or any other such personnel that interacts with the CAS system 100. For example, actor 112 can record data from the CAS system 100, configure/update one or more attributes of the CAS system 100, review past performance of the CAS system 100, repair the CAS system 100, and/or the like including combinations and/or multiples thereof.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis). During each step, certain surgical instruments 108 (e.g., forceps) are used to achieve a specific objective by performing one or more surgical actions. In addition, a particular anatomical structure of the patient may be the target of the surgical action(s).

The video recording system 104 includes one or more cameras 105, such as operating room cameras, endoscopic cameras, and/or the like including combinations and/or multiples thereof. The cameras 105 capture video data of the surgical procedure being performed. The video recording system 104 includes one or more video capture devices that can include cameras 105 placed in the surgical room to capture events surrounding (i.e., outside) the patient being operated upon. The video recording system 104 further includes cameras 105 that are passed inside (e.g., endoscopic cameras) the patient 110 to capture endoscopic data. The endoscopic data provides video and images of the surgical procedure.

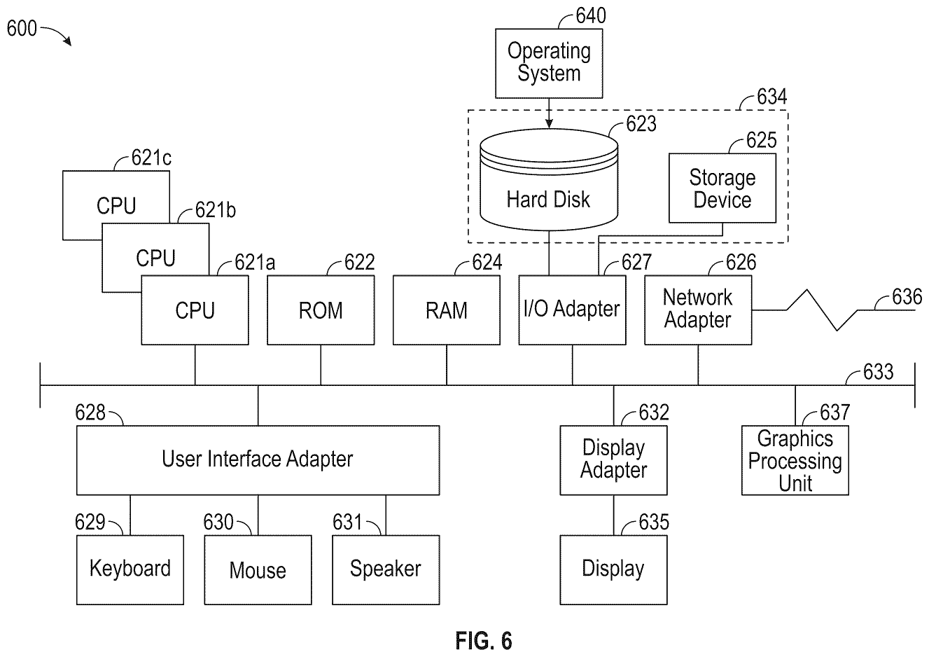

The computing system 102 includes one or more memory devices, one or more processors, a user interface device, among other components. All or a portion of the computing system 102 shown in FIG. 1 can be implemented for example, by all or a portion of computer system 600 of FIG. 6. Computing system 102 can execute one or more computer-executable instructions. The execution of the instructions facilitates the computing system 102 to perform one or more methods, including those described herein. The computing system 102 can communicate with other computing systems via a wired and/or a wireless network. In one or more examples, the computing system 102 includes one or more trained machine learning models that can detect and/or predict features of/from the surgical procedure that is being performed or has been performed earlier. Features can include structures, such as anatomical structures, surgical instruments 108 in the captured video of the surgical procedure. Features can further include events, such as phases and/or actions in the surgical procedure. Features that are detected can further include the actor 112 and/or patient 110. Based on the detection, the computing system 102, in one or more examples, can provide recommendations for subsequent actions to be taken by the actor 112. Alternatively, or in addition, the computing system 102 can provide one or more reports based on the detections. The detections by the machine learning models can be performed in an autonomous or semi-autonomous manner.

The machine learning models can include artificial neural networks, such as deep neural networks, convolutional neural networks, recurrent neural networks, vision transformers, encoders, decoders, or any other type of machine learning model. The machine learning models can be trained in a supervised, unsupervised, or hybrid manner. The machine learning models can be trained to perform detection and/or prediction using one or more types of data acquired by the CAS system 100. For example, the machine learning models can use the video data captured via the video recording system 104. Alternatively, or in addition, the machine learning models use the surgical instrumentation data from the surgical instrumentation system 106. In yet other examples, the machine learning models use a combination of video data and surgical instrumentation data.

Additionally, in some examples, the machine learning models can also use audio data captured during the surgical procedure. The audio data can include sounds emitted by the surgical instrumentation system 106 while activating one or more surgical instruments 108. Alternatively, or in addition, the audio data can include voice commands, snippets, or dialog from one or more actors 112. The audio data can further include sounds made by the surgical instruments 108 during their use.

In one or more examples, the machine learning models can detect surgical actions, surgical phases, anatomical structures, surgical instruments, and various other features from the data associated with a surgical procedure. The detection can be performed in real-time in some examples. Alternatively, or in addition, the computing system 102 analyzes the surgical data, i.e., the various types of data captured during the surgical procedure, in an offline manner (e.g., post-surgery). In one or more examples, the machine learning models detect surgical phases based on detecting some of the features, such as the anatomical structure, surgical instruments, and/or the like including combinations and/or multiples thereof.

A data collection system 150 can be employed to store the surgical data, including the video(s) captured during the surgical procedures. The data collection system 150 includes one or more storage devices 152. The data collection system 150 can be a local storage system, a cloud-based storage system, or a combination thereof. Further, the data collection system 150 can use any type of cloud-based storage architecture, for example, public cloud, private cloud, hybrid cloud, and/or the like including combinations and/or multiples thereof. In some examples, the data collection system can use a distributed storage, i.e., the storage devices 152 are located at different geographic locations. The storage devices 152 can include any type of electronic data storage media used for recording machine-readable data, such as semiconductor-based, magnetic-based, optical-based storage media, and/or the like including combinations and/or multiples thereof. For example, the data storage media can include flash-based solid-state drives (SSDs), magnetic-based hard disk drives, magnetic tape, optical discs, and/or the like including combinations and/or multiples thereof.

In one or more examples, the data collection system 150 can be part of the video recording system 104, or vice-versa. In some examples, the data collection system 150, the video recording system 104, and the computing system 102, can communicate with each other via a communication network, which can be wired, wireless, or a combination thereof. The communication between the systems can include the transfer of data (e.g., video data, instrumentation data, and/or the like including combinations and/or multiples thereof), data manipulation commands (e.g., browse, copy, paste, move, delete, create, compress, and/or the like including combinations and/or multiples thereof), data manipulation results, and/or the like including combinations and/or multiples thereof. In one or more examples, the computing system 102 can manipulate the data already stored/being stored in the data collection system 150 based on outputs from the one or more machine learning models (e.g., phase detection, anatomical structure detection, surgical tool detection, and/or the like including combinations and/or multiples thereof). Alternatively, or in addition, the computing system 102 can manipulate the data already stored/being stored in the data collection system 150 based on information from the surgical instrumentation system 106.

In one or more examples, the video captured by the video recording system 104 is stored on the data collection system 150. In some examples, the computing system 102 curates parts of the video data being stored on the data collection system 150. In some examples, the computing system 102 filters the video captured by the video recording system 104 before it is stored on the data collection system 150. Alternatively, or in addition, the computing system 102 filters the video captured by the video recording system 104 after it is stored on the data collection system 150.

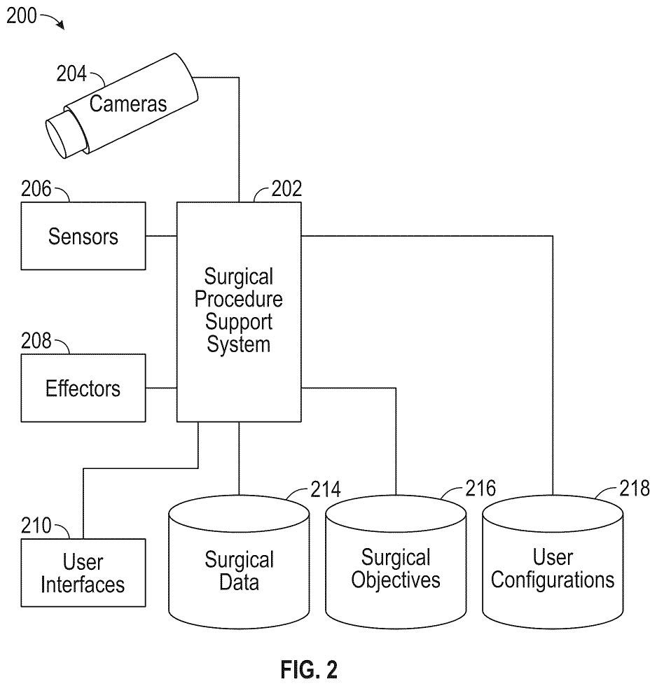

Turning now to FIG. 2, a surgical procedure system 200 is generally shown according to one or more embodiments described herein. The example of FIG. 2 depicts a surgical procedure support system 202 that can include or may be coupled to the CAS system 100 of FIG. 1. The surgical procedure support system 202 can acquire image or video data using one or more cameras 204. The surgical procedure support system 202 can also interface with one or more sensors 206 and/or one or more effectors 208. The sensors 206 may be associated with surgical support equipment and/or patient monitoring. The effectors 208 can be robotic components or other equipment controllable through the surgical procedure support system 202. The surgical procedure support system 202 can also interact with one or more user interfaces 210, such as various input and/or output devices. The surgical procedure support system 202 can store, access, and/or update surgical data 214 associated with a training dataset and/or live data as a surgical procedure is being performed on patient 110 of FIG. 1. The surgical procedure support system 202 can store, access, and/or update surgical objectives 216 to assist in training and guidance for one or more surgical procedures. User configurations 218 can track and store user preferences.

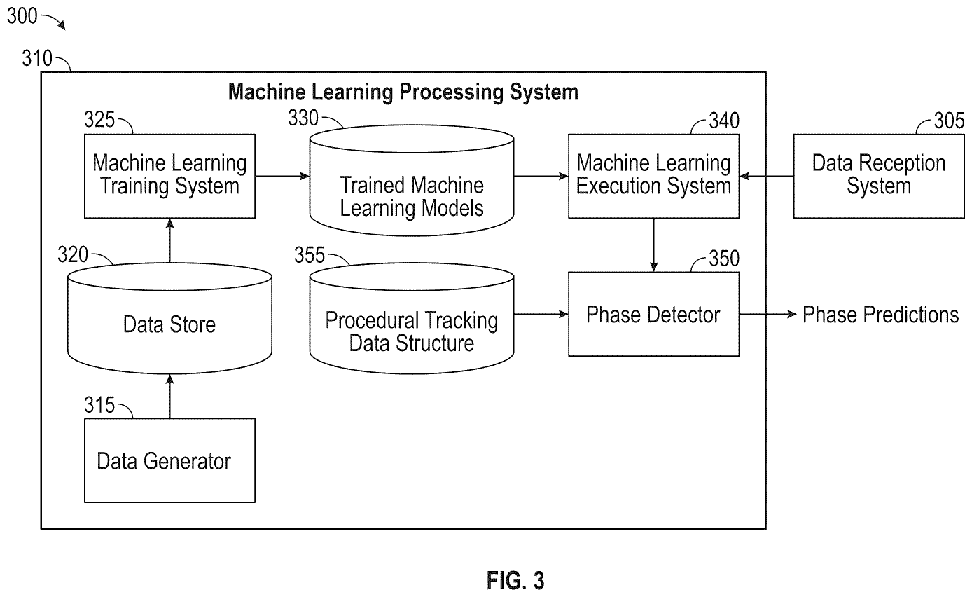

Turning now to FIG. 3, a system 300 for analyzing video and data is generally shown according to one or more embodiments described herein. In accordance with aspects, the video and data is captured from video recording system 104 of FIG. 1. The analysis can result in predicting features that include surgical phases and structures (e.g., instruments, anatomical structures, and/or the like including combinations and/or multiples thereof) in the video data using machine learning. System 300 can be the computing system 102 of FIG. 1, or a part thereof in one or more examples. System 300 uses data streams in the surgical data to identify procedural states according to some aspects.

System 300 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed. Alternatively, or in addition, the data reception system 305 can receive or access surgical data in an offline manner, for example, by accessing data that is stored in the data collection system 150 of FIG. 1.

System 300 further includes a machine learning processing system 310 that processes the surgical data using one or more machine learning models to identify one or more features, such as surgical phase, instrument, anatomical structure, and/or the like including combinations and/or multiples thereof, in the surgical data. It will be appreciated that machine learning processing system 310 can include one or more devices (e.g., one or more servers), each of which can be configured to include part or all of one or more of the depicted components of the machine learning processing system 310. In some instances, a part or all of the machine learning processing system 310 is cloud-based and/or remote from an operating room and/or physical location corresponding to a part or all of data reception system 305. It will be appreciated that several components of the machine learning processing system 310 are depicted and described herein. However, the components are just one example structure of the machine learning processing system 310, and that in other examples, the machine learning processing system 310 can be structured using a different combination of the components. Such variations in the combination of the components are encompassed by the technical solutions described herein.

The machine learning processing system 310 includes a machine learning training system 325, which can be a separate device (e.g., server) that stores its output as one or more trained machine learning models 330. The machine learning models 330 are accessible by a machine learning execution system 340. The machine learning execution system 340 can be separate from the machine learning training system 325 in some examples. In other words, in some aspects, devices that “train” the models are separate from devices that “infer,” i.e., perform real-time processing of surgical data using the trained machine learning models 330.

Machine learning processing system 310, in some examples, further includes a data generator 315 to generate simulated surgical data, such as a set of virtual images, or record the video data from the video recording system 104, to generate trained machine learning models 330. Data generator 315 can access (read/write) a data store 320 to record data, including multiple images and/or multiple videos. The images and/or videos can include images and/or videos collected during one or more procedures (e.g., one or more surgical procedures). For example, the images and/or video may have been collected by a user device worn by the actor 112 of FIG. 1 (e.g., surgeon, surgical nurse, anesthesiologist, and/or the like including combinations and/or multiples thereof) during the surgery, a non-wearable imaging device located within an operating room, an endoscopic camera inserted inside the patient 110 of FIG. 1, and/or the like including combinations and/or multiples thereof. The data store 320 is separate from the data collection system 150 of FIG. 1 in some examples. In other examples, the data store 320 is part of the data collection system 150.

Each of the images and/or videos recorded in the data store 320 for performing training (e.g., generating the machine learning models 330) can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, and/or the like including combinations and/or multiples thereof). Further, the other data can include image-segmentation data that identifies and/or characterizes one or more objects (e.g., tools, anatomical objects, and/or the like including combinations and/or multiples thereof) that are depicted in the image or video. The characterization can indicate the position, orientation, or pose of the object in the image. For example, the characterization can indicate a set of pixels that correspond to the object and/or a state of the object resulting from a past or current user handling. Localization can be performed using a variety of techniques for identifying objects in one or more coordinate systems.

The machine learning training system 325 uses the recorded data in the data store 320, which can include the simulated surgical data (e.g., set of virtual images) and/or actual surgical data to generate the trained machine learning models 330. The trained machine learning models 330 can be defined based on a type of model and a set of hyperparameters (e.g., defined based on input from a client device). The trained machine learning models 330 can be configured based on a set of parameters that can be dynamically defined based on (e.g., continuous or repeated) training (i.e., learning, parameter tuning). Machine learning training system 325 can use one or more optimization algorithms to define the set of parameters to minimize or maximize one or more loss functions. The set of (learned) parameters can be stored as part of the trained machine learning models 330 using a specific data structure for a particular trained machine learning model of the trained machine learning models 330. The data structure can also include one or more non-learnable variables (e.g., hyperparameters and/or model definitions).

Machine learning execution system 340 can access the data structure(s) of the trained machine learning models 330 and accordingly configure the trained machine learning models 330 for inference (e.g., prediction, classification, and/or the like including combinations and/or multiples thereof). The trained machine learning models 330 can include, for example, a fully convolutional network adaptation, an adversarial network model, an encoder, a decoder, or other types of machine learning models. The type of the trained machine learning models 330 can be indicated in the corresponding data structures. The trained machine learning models 330 can be configured in accordance with one or more hyperparameters and the set of learned parameters.

The trained machine learning models 330, during execution, receive, as input, surgical data to be processed and subsequently generate one or more inferences according to the training. For example, the video data captured by the video recording system 104 of FIG. 1 can include data streams (e.g., an array of intensity, depth, and/or RGB values) for a single image or for each of a set of frames (e.g., including multiple images or an image with sequencing data) representing a temporal window of fixed or variable length in a video. The video data that is captured by the video recording system 104 can be received by the data reception system 305, which can include one or more devices located within an operating room where the surgical procedure is being performed. Alternatively, the data reception system 305 can include devices that are located remotely, to which the captured video data is streamed live during the performance of the surgical procedure. Alternatively, or in addition, the data reception system 305 accesses the data in an offline manner from the data collection system 150 or from any other data source (e.g., local or remote storage device).

The data reception system 305 can process the video and/or data received. The processing can include decoding when a video stream is received in an encoded format such that data for a sequence of images can be extracted and processed. The data reception system 305 can also process other types of data included in the input surgical data. For example, the surgical data can include additional data streams, such as audio data, RFID data, textual data, measurements from one or more surgical instruments/sensors, and/or the like including combinations and/or multiples thereof, that can represent stimuli/procedural states from the operating room. The data reception system 305 synchronizes the different inputs from the different devices/sensors before inputting them in the machine learning processing system 310.

The trained machine learning models 330, once trained, can analyze the input surgical data, and in one or more aspects, predict and/or characterize features (e.g., structures) included in the video data included with the surgical data. The video data can include sequential images and/or encoded video data (e.g., using digital video file/stream formats and/or codecs, such as MP4, MOV, AVI, WEBM, AVCHD, OGG, and/or the like including combinations and/or multiples thereof). The prediction and/or characterization of the features can include segmenting the video data or predicting the localization of the structures with a probabilistic heatmap. In some instances, the one or more trained machine learning models 330 include or are associated with a preprocessing or augmentation (e.g., intensity normalization, resizing, cropping, and/or the like including combinations and/or multiples thereof) that is performed prior to segmenting the video data. An output of the one or more trained machine learning models 330 can include image-segmentation or probabilistic heatmap data that indicates which (if any) of a defined set of structures are predicted within the video data, a location and/or position and/or pose of the structure(s) within the video data, and/or state of the structure(s). The location can be a set of coordinates in an image/frame in the video data. For example, the coordinates can provide a bounding box. The coordinates can provide boundaries that surround the structure(s) being predicted. The trained machine learning models 330, in one or more examples, are trained to perform higher-level predictions and tracking, such as predicting a phase of a surgical procedure and tracking one or more surgical instruments used in the surgical procedure.

While some techniques for predicting a surgical phase (“phase”) in the surgical procedure are described herein, it should be understood that any other technique for phase prediction can be used without affecting the aspects of the technical solutions described herein. In some examples, the machine learning processing system 310 includes a phase detector 350 that uses the trained machine learning models 330 to identify a phase within the surgical procedure (“procedure”). Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by actor 112. The procedural tracking data structure 355 identifies a set of potential phases that can correspond to a part of the specific type of procedure as “phase predictions.”

In some examples, the procedural tracking data structure 355 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 355 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, and/or the like including combinations and/or multiples thereof), pre-condition (e.g., lesions, polyps, and/or the like including combinations and/or multiples thereof). In some examples, the trained machine learning models 330 are trained to detect an “abnormal condition,” such as hemorrhaging, arrhythmias, blood vessel abnormality, and/or the like including combinations and/or multiples thereof.

Each node within the procedural tracking data structure 355 can identify one or more characteristics of the phase corresponding to that node. The characteristics can include visual characteristics. In some instances, the node identifies one or more tools that are typically in use or available for use (e.g., on a tool tray) during the phase. The node also identifies one or more roles of people who are typically performing a surgical task, a typical type of movement (e.g., of a hand or tool), and/or the like including combinations and/or multiples thereof. Thus, phase detector 350 can use the segmented data generated by machine learning execution system 340 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, and/or the like including combinations and/or multiples thereof).

The phase detector 350 outputs the phase prediction associated with a portion of the video data that is analyzed by the machine learning processing system 310. The phase prediction is associated with the portion of the video data by identifying a start time and an end time of the portion of the video that is analyzed by the machine learning execution system 340. The phase prediction that is output can include segments of the video where each segment corresponds to and includes an identity of a surgical phase as detected by the phase detector 350 based on the output of the machine learning execution system 340. Further, the phase prediction, in one or more examples, can include additional data dimensions, such as, but not limited to, identities of the structures (e.g., instrument, anatomy, and/or the like including combinations and/or multiples thereof) that are identified by the machine learning execution system 340 in the portion of the video that is analyzed. The phase prediction can also include a confidence score of the prediction. Other examples can include various other types of information in the phase prediction that is output.

It should be noted that although some of the drawings depict endoscopic videos being analyzed, the technical solutions described herein can be applied to analyze video and image data captured by cameras that are not endoscopic (i.e., cameras external to the patient's body) when performing open surgeries (i.e., not laparoscopic surgeries). For example, the video and image data can be captured by cameras that are mounted on one or more personnel in the operating room (e.g., surgeon). Alternatively, or in addition, the cameras can be mounted on surgical instruments, walls, or other locations in the operating room. Alternatively, or in addition, the video can be images captured by other imaging modalities, such as ultrasound.

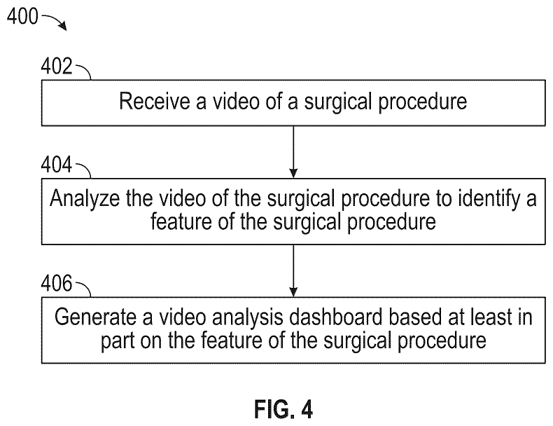

Turning now to FIG. 4, a flow diagram of a method 400 is shown according to one or more embodiments described herein. The method 400 can be performed by any suitable processing system, such as the computing system 102, the processing system 600 of FIG. 6, and/or the like including combinations and/or multiples thereof.

At block 402, a system, such as the computing system 102 and/or the processing system 600, receives a video of a surgical procedure. The surgical procedure can be any type of surgery, such as but not limited to cataract surgery, laparoscopic cholecystectomy, endoscopic endonasal transsphenoidal approach (eTSA) to resection of pituitary adenomas, or any other surgical procedure. The video can be captured by any suitable system or device, such as the video recording system 104 and/or the surgical procedure system 200. According to one or more embodiments, receiving the video can include any form of accessing at least a portion of the video.

At block 404, the system analyzes the video of the surgical procedure to predict features of the surgical procedure. As an example, the system 300 can be used to analyze the video of the surgical procedure as described herein. The analysis can result in predicting features that include surgical phases and structures (e.g., instruments, anatomical structures, and/or the like including combinations and/or multiples thereof) in the video data using machine learning. For example, machine learning models can be used to detect surgical phases based on detecting features, such as the anatomical structure, surgical instruments, and/or the like including combinations and/or multiples thereof.

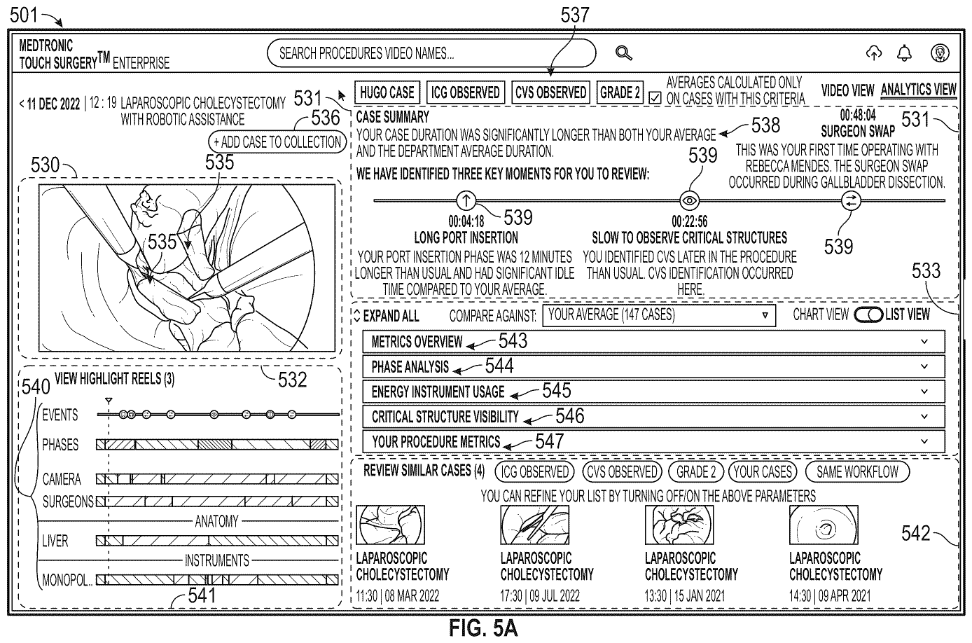

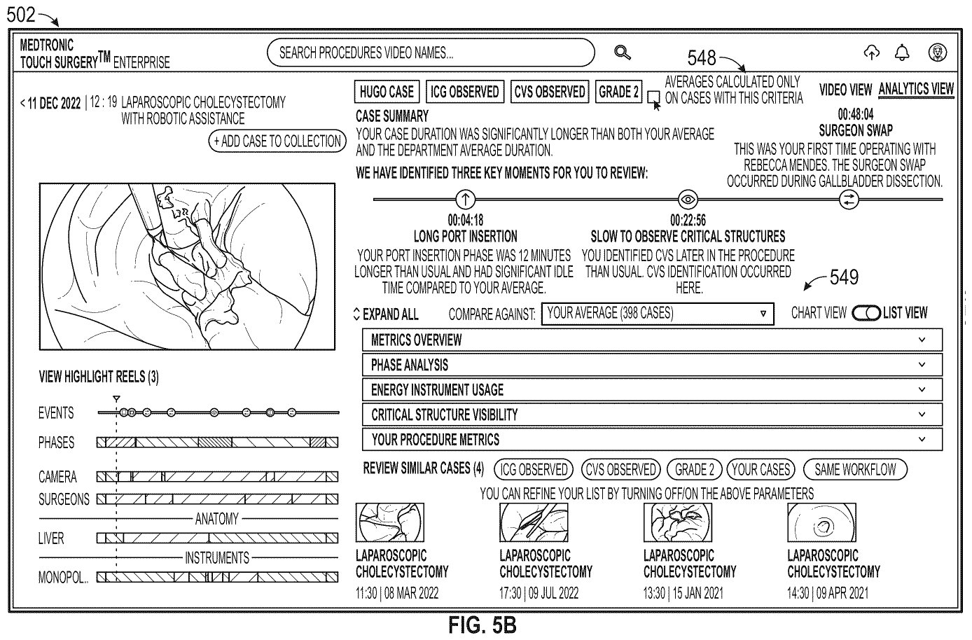

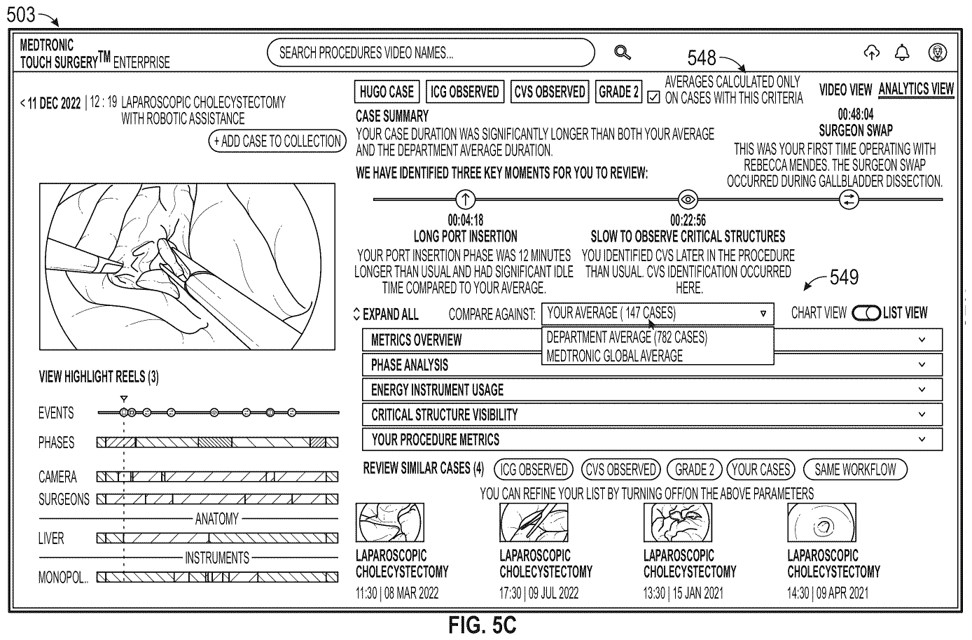

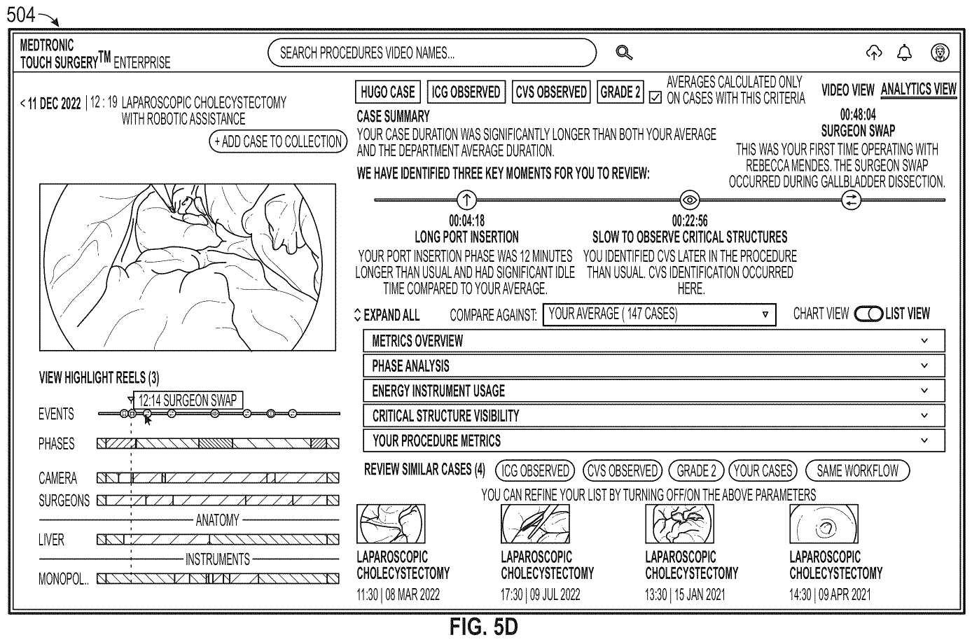

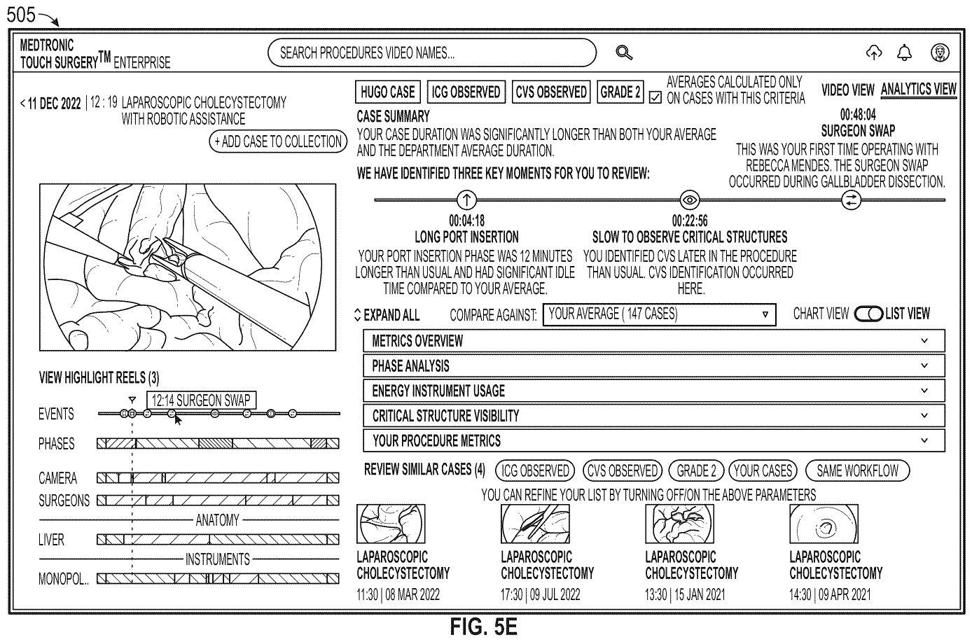

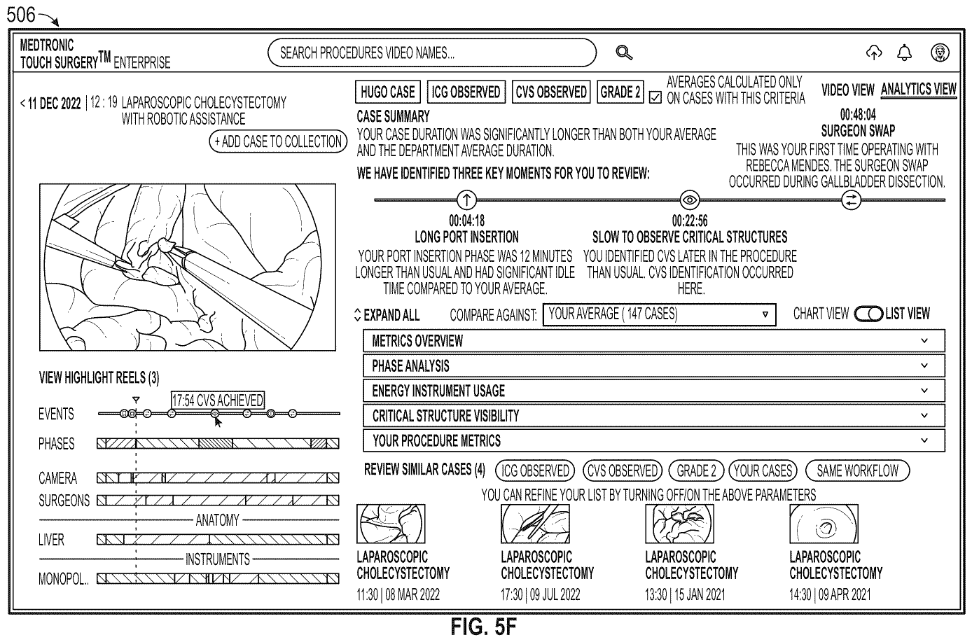

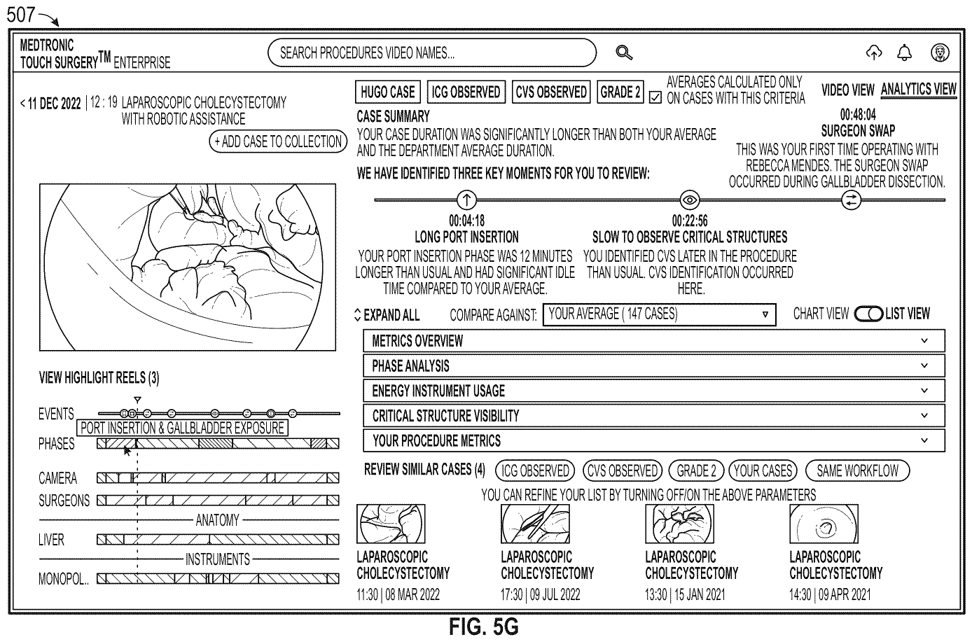

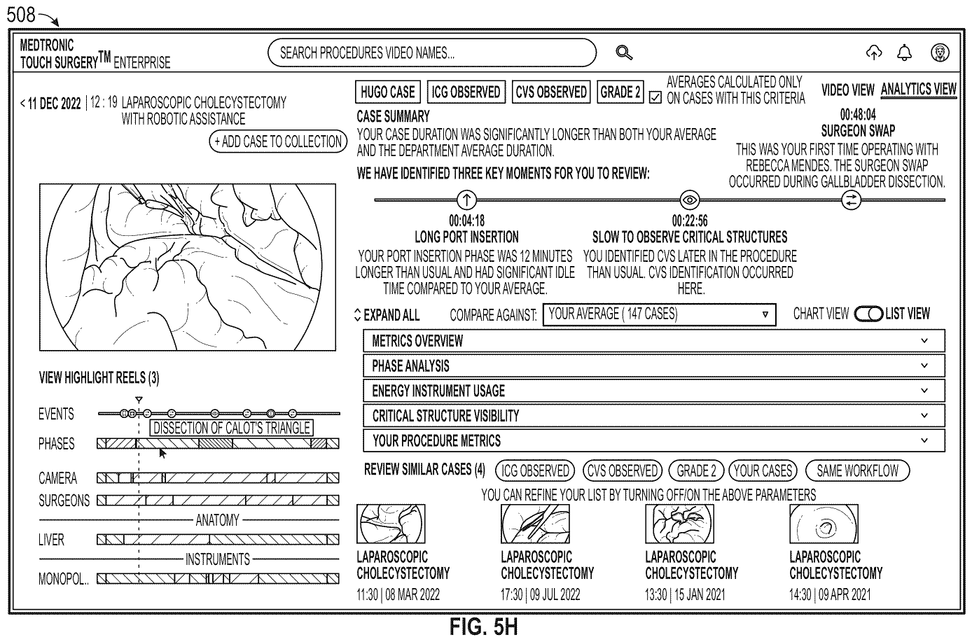

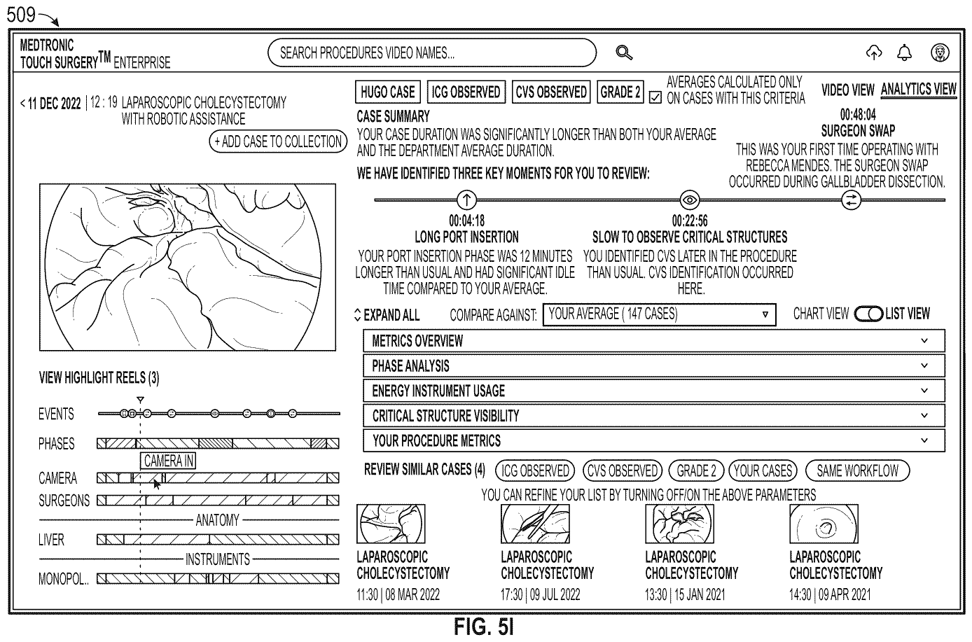

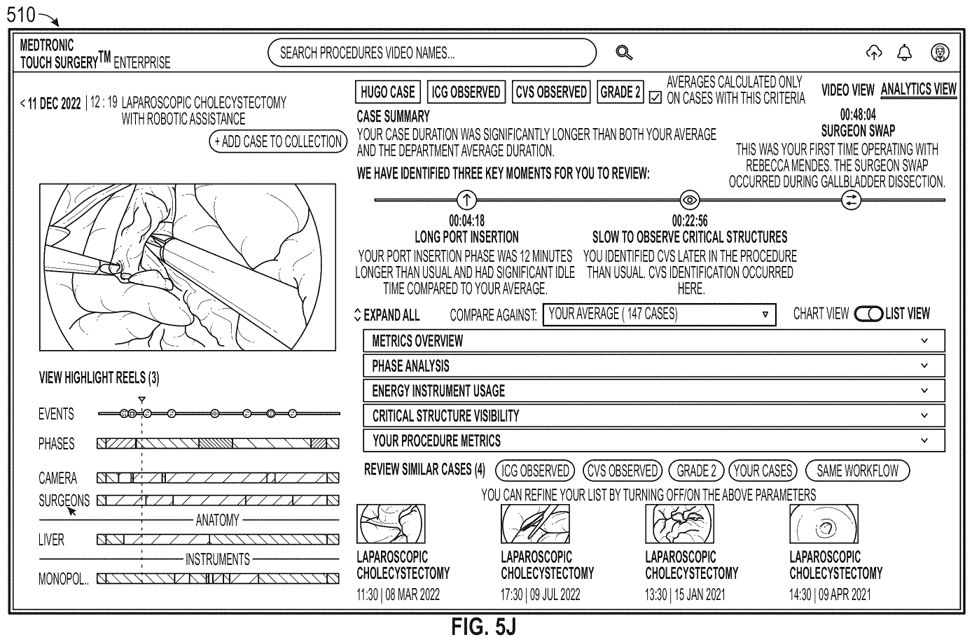

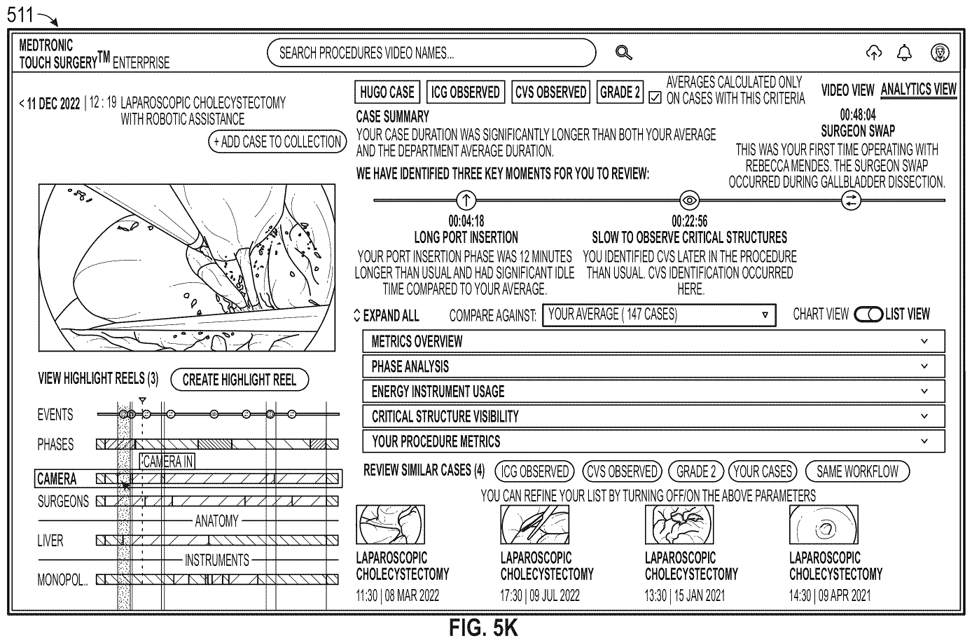

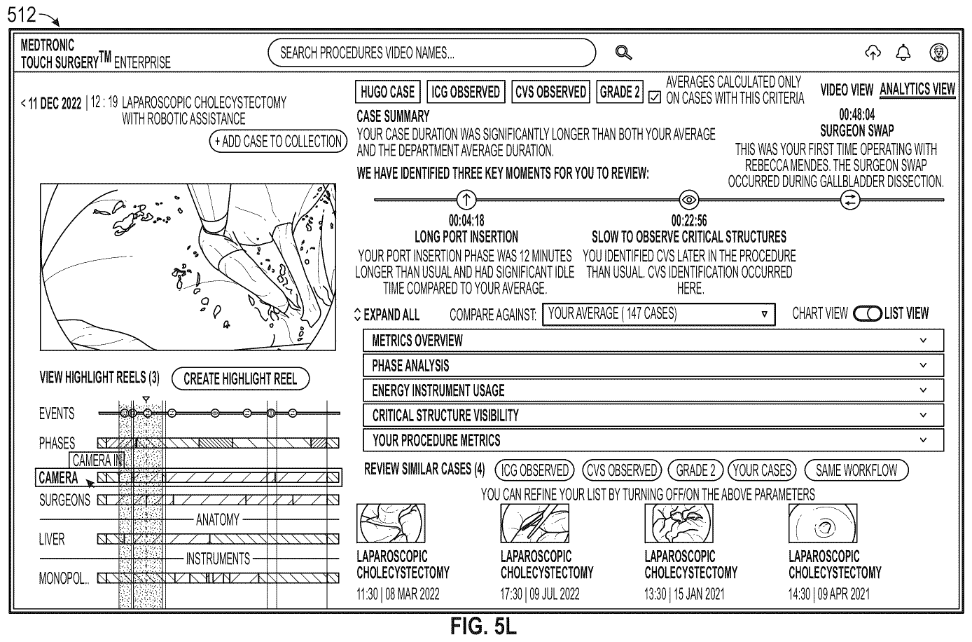

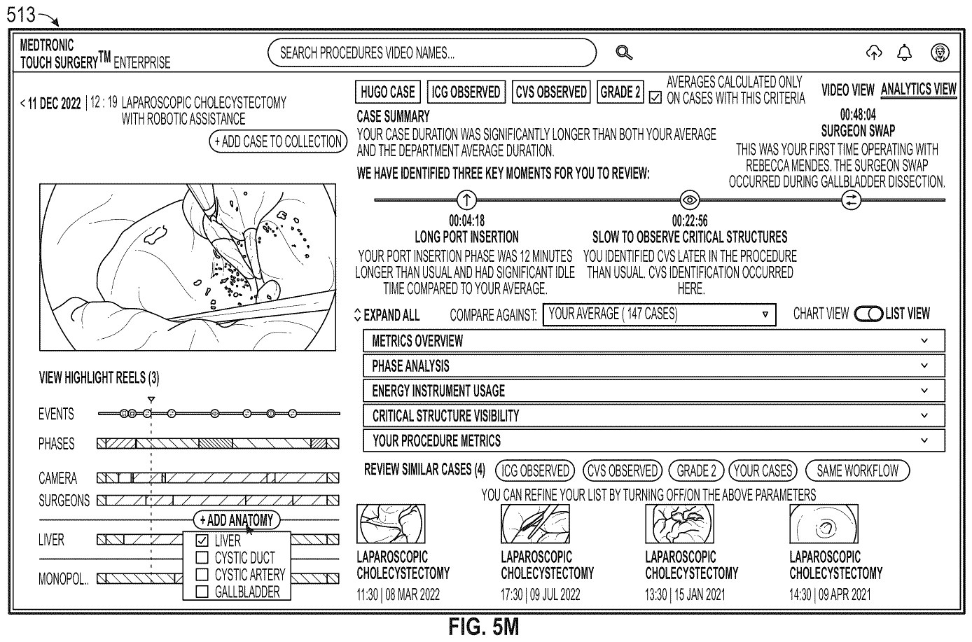

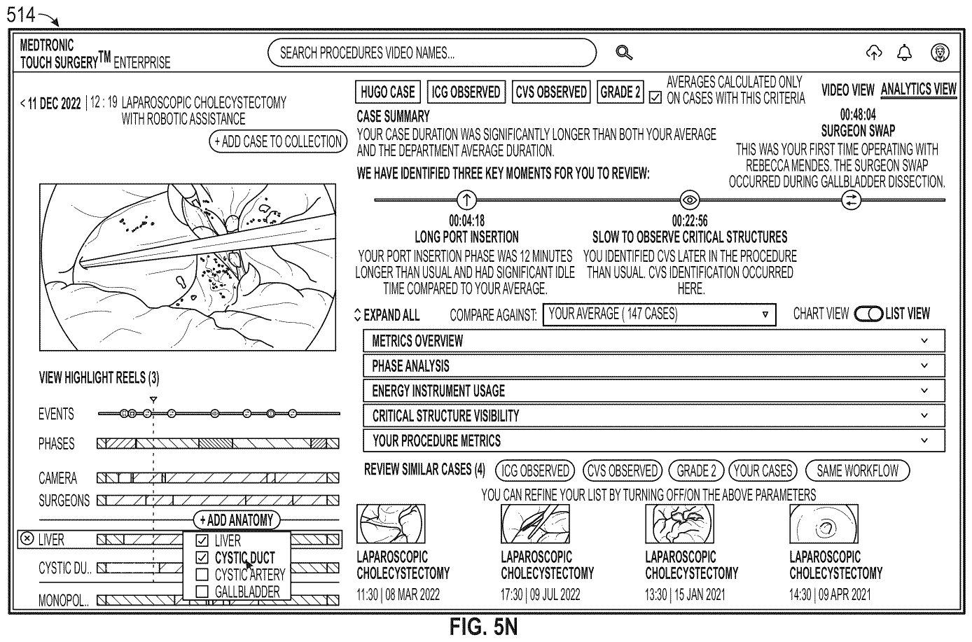

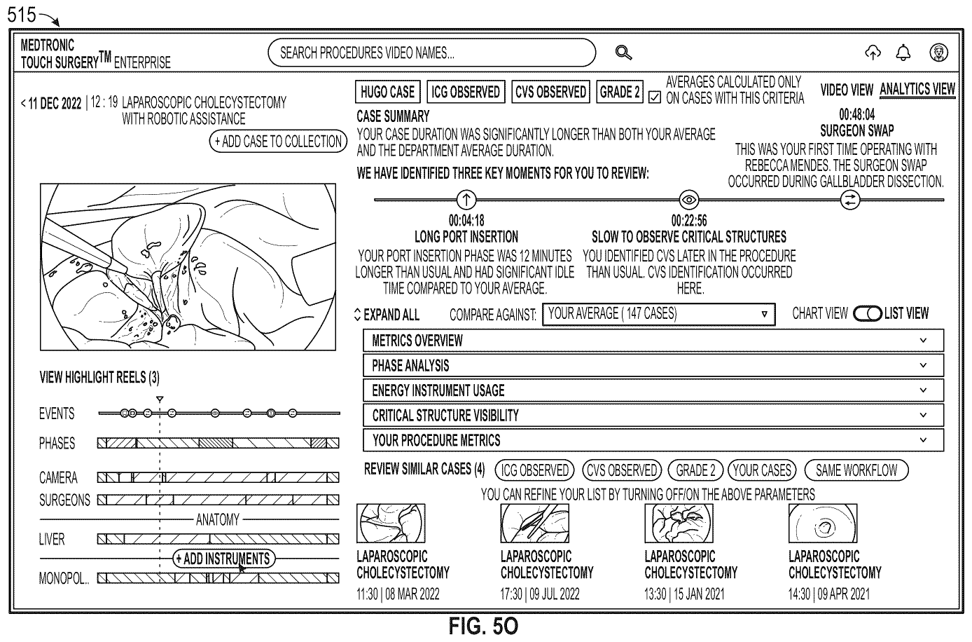

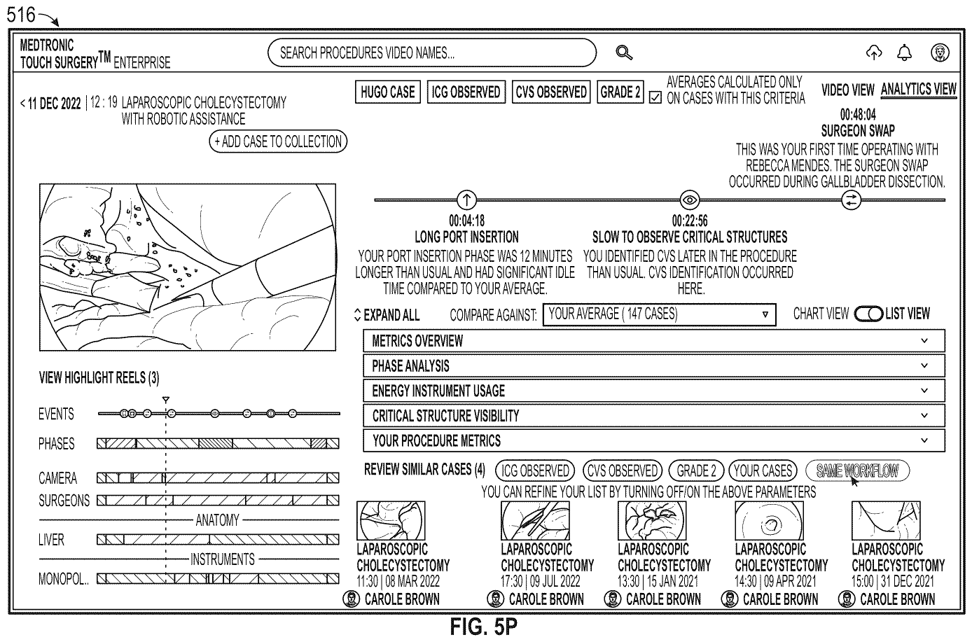

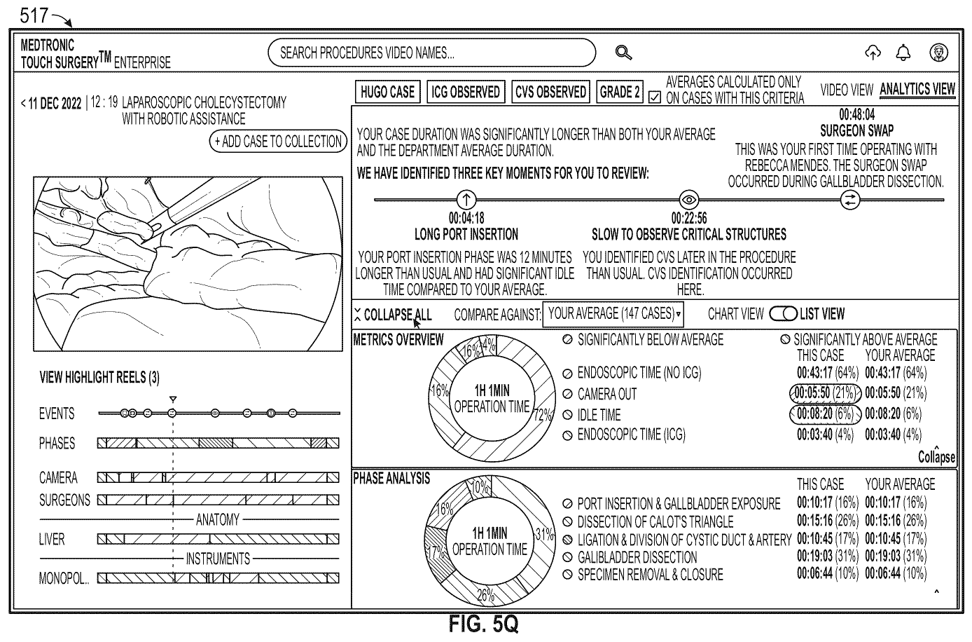

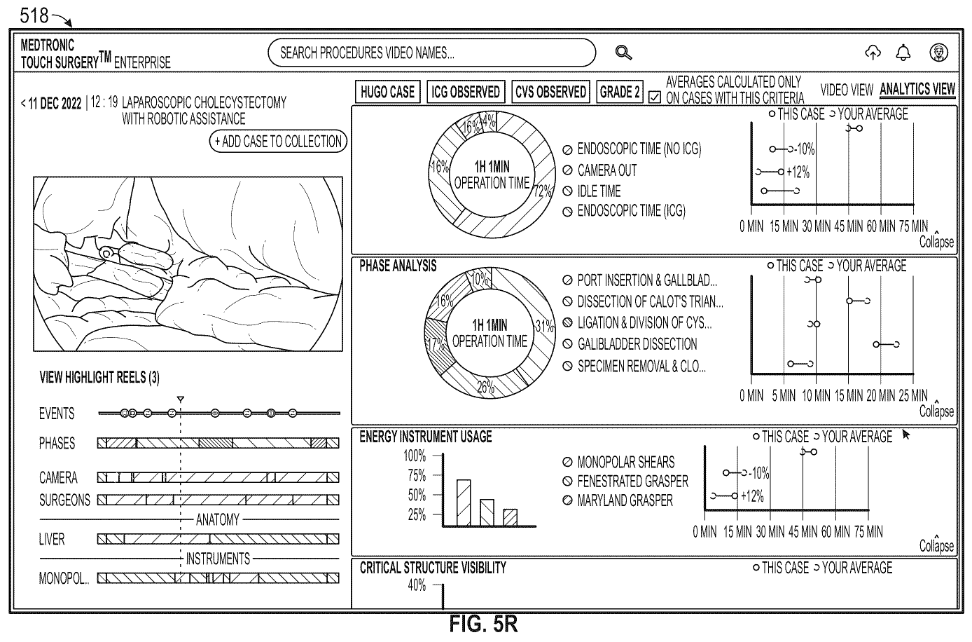

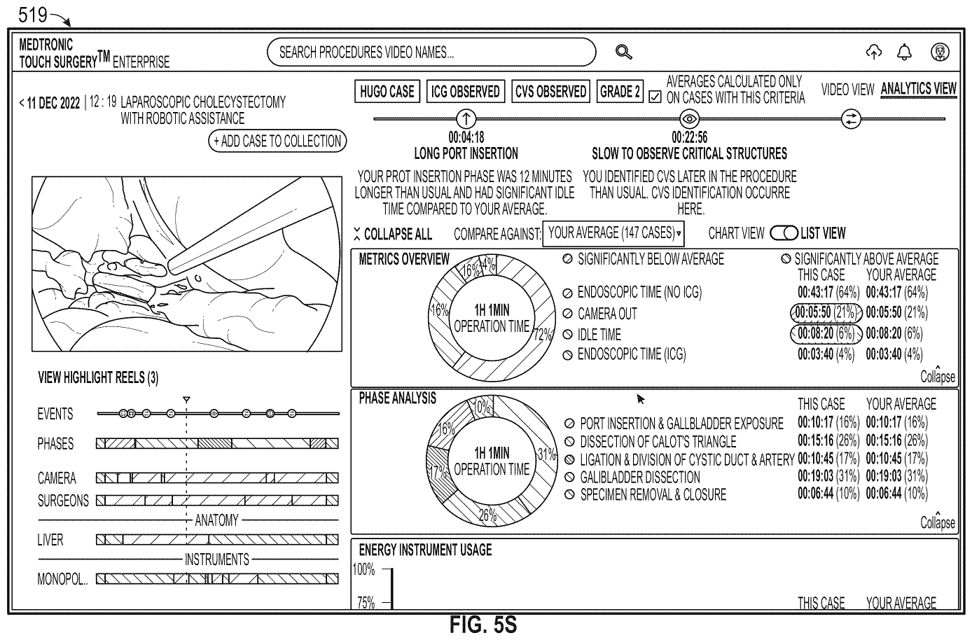

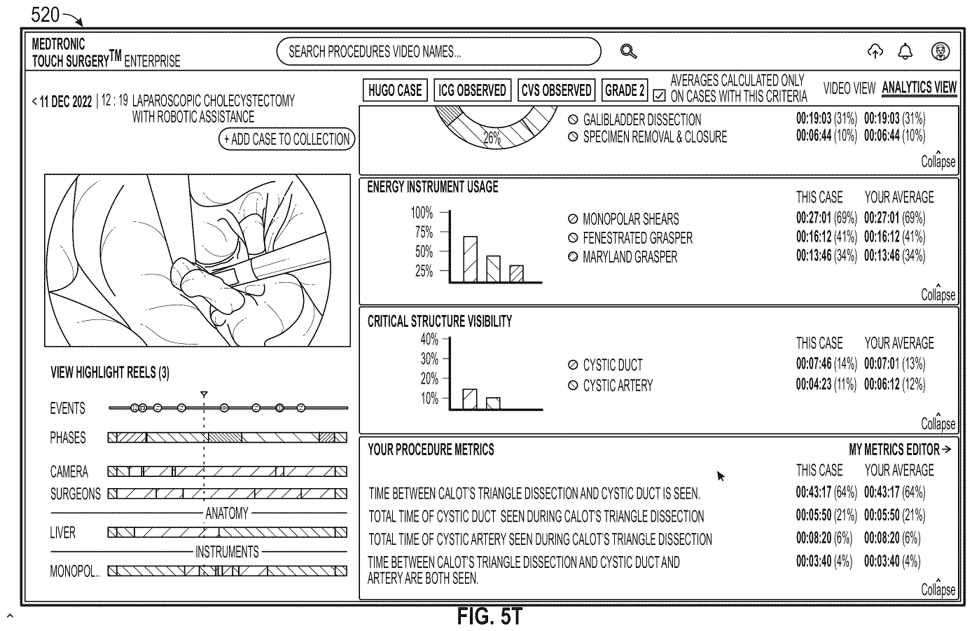

At block 406, the processing system generates a video analysis dashboard based at least in part on the feature of the surgical procedure from block 404. Examples of the video analysis dashboard 501-520 are now described in more detail with reference to FIGS. 5A-5T but the examples of these figures are not intended to limit the claims.

According to an embodiment with reference to FIG. 5A, the video analysis dashboard 501 includes a video region 530 to display the video of the surgical procedure, a case summary region 531 to display a case summary of the surgical procedure, a timeline reel region 532 to display highlights of the surgical procedure, and an analytics region 533 to display analytics of the surgical procedure.

The video can include augmented reality (AR) elements 535 associated with certain features of the video, such as anatomy, surgical instruments, and/or the like, including combinations and/or multiples thereof. According to one or more embodiments described herein, the video analysis dashboard 501 can include an option for downloading the video with the AR elements overlaid or with no AR elements overlaid.

According to an embodiment, the video analysis dashboard 501 can provide for adding a video to a collection (e.g., folder) for categorizing videos into, for example, conference videos, training, and/or the like, including combinations and/or multiples thereof. For example, the video analysis dashboard 501 can include an add case to collection button 536.

According to an embodiment, the video analysis dashboard 501 can provide for viewing case tags 537 added by machine learning or manual annotation and set comparison averages for cases which follow the same or similar criteria. Some non-limiting examples of case tags are as follows: Grade 1-4 or Standard/Complex, Procedure type (e.g., subtotal for lap chloe procedure), indocyanine green dye (ICG) observed or can reference another surgical technique, cardiovascular system (CVS) observed, robot-assisted surgery (RAS), trainee case, incomplete video (e.g., start/end missing), elective/non-elective, conversion to open, research study tag (e.g., the surgeon can reference that the case is part of study X), alternative imaging modality, patient metadata (e.g., body mass index, gender, comorbidity), and/or the like, including combinations and/or multiples thereof.

According to an embodiment, the video analysis dashboard 501 can provide for viewing a case summary sentence 538 generated by machine learning and analytics.

According to an embodiment, the video analysis dashboard 501 can provide for viewing key surgical moments (or “key moments”) 539 generated automatically by machine learning and analytics. For example, a viewer (e.g., the surgeon) of the video analysis dashboard 501 can jump to specific points in the surgical video by clicking on a moment of identified key surgical moments. According to one or more embodiments described herein, the key moments can be identified using a machine learning algorithm and/or a statistical analysis, such as an outlier analysis. Some non-limiting examples of key moments are as follows:
Phase X was significantly longer/shorter compared to the surgeon's average
The surgeon transitioned from Phase X to Phase Y, which may indicate an unusual phase sequence in the surgeon's workflows (e.g., highlighting a potential new technique)
A surgeon swap happened with a second surgeon the surgeon is operating with for the first time.
The surgeon observed a critical structure earlier/later in the case than usual.
The surgeon used a new instrument for the first time from this timepoint.
The surgeon annotated X at this timestamp during the case (e.g., highlighting to the surgeon a potential key moment that the surgeon identified during the surgery).
The surgeon toggled between phase A and phase B more than X times during this procedure starting from this timepoint (e.g., highlighting a potentially complex part of the case that the surgeon may want to review).
The surgeon had a new trainee operating with the surgeon from this point of the procedure (e.g., highlighting part of the case they might want to review their trainee performance).

According to an embodiment, the key surgical moments 539 and/or the analytics displayed in the analytics region 533 described herein are determined relative to other data, such as data for the surgeon for the same or similar surgical procedures, data for other surgeons within the department for the same or similar surgical procedures, and/or global data for other surgeons for the same or similar surgical procedures. That is, the data for the key surgical moments 539 and/or the analytics region 533 is based on an average, such as the for a particular surgeon who performed the surgical procedure, the average for a group of surgeons associated with the particular surgeon, or the global average for a global population of surgeons. The video analysis dashboards 502 and 533 of FIGS. 5B and 5C respectively show options for averages calculated only on cases with this criteria 548 and for which group (e.g., only the surgeon, other surgeons in the department, a global population of surgeons, and/or the like, including combinations and/or multiples thereof) the data is compared against 549. According to one or more embodiments described herein, data for the analytics region is based on a statistical analysis of the video (e.g., determining an average, a standard deviation and outlier detection).

According to an embodiment, the video analysis dashboard 501 can provide for viewing timelines 540 for the video for camera, phase, surgeon, instrument, anatomy, and/or events within the timeline reel region 532. For example, a visual indicator, such as a line 541, moves along timelines 540 as the video plays. A viewer (e.g., the surgeon) of the video analysis dashboard 501 can select a point on a timeline to display grid lines through the other timelines to provide for alignment to be easily seen. A viewer of the video analysis dashboard 501 can also select segments in the timeline to jump to a specific point in video (see, e.g., the video analysis dashboards 504-510 of FIGS. 5D-5J. Some non-limiting examples of events are as follows: bleeding, bile duct spillage, surgeon swap, the surgeon's own events annotated as well as bookmarks and comments, critical structure observed, swabs inserted, complication, and/or the like, including combinations and/or multiples thereof. According to an embodiment, the video analysis dashboards 513 and 514 of FIGS. 5L and 5M show that an anatomy can be added (e.g., a liver, a cystic duct, a cystic artery, a gallbladder) to the timelines 540. Similarly, as shown in the video analysis dashboard 515 of FIG. 5N, an instrument can be added to the timelines 540. According to one or more embodiments, event annotation can be tracked as metadata or additional file information that aligns with the video and may be editable through a separate interface.

According to an embodiment, the video analysis dashboard 501 can provide for creating highlight reels by clicking on one or more segments within the timeline reel region 532. A highlight reel is a sequence of video clips. For example, a surgeon wants to create a reel of parts of the surgical procedure, for a specific phase and using a specific device. In such cases, the surgeon can click on desired segments for the specific phase and using a specific device, and a highlight reel is created (see, e.g., the video analysis dashboards 511 and 512 of FIGS. 5K and 5L). According to an embodiment, the video analysis dashboard 501 can include a download button to download the highlight reel. According to one or more embodiments, highlight reels can be created from unmodified video content or may include AR element 535 overlays and/or other information associated with the selected portions of video.

According to an embodiment, the video analysis dashboard 501 includes the analytics region 533, which provides for viewing case analytics as a metrics overview 543, instrument breakdown as energy instrument usage 545, phase breakdown as phase analysis 544, critical structure breakdown as critical structure visibility 546, and/or procedure metrics analytics as your procedure metrics 547 in the analytics region 533. According to one or more embodiments described herein, a user can create or specify metrics particular to the user, referred to as user created metrics. More detailed views of portions of the analytics region 533 are shown in the video analysis dashboards 517-520 of FIGS. 5Q-5T. According to embodiments, the analytics region 533 can be displayed as a list view (e.g., FIGS. 5Q, 5S, 5T) or a chart view (e.g., FIG. 5R). The list view includes, for example, times for different events (e.g., endoscope time (no ICG), camera out, idle time, endoscope time (ICG) for the particular surgical procedure against an average, which as described herein can be for the surgeon, for other surgeons within the department, for a global population of surgeons, total endoscopic time, the number of camera in/out events, and/or the like, including combinations and/or multiples thereof. The chart view shows charts that plot the values for the list view in a graphical format as shown. Other configurations of chart views are also possible. According to one or more embodiments, user interaction with the analytics region 533 can selectively expand any combination of analytic categories 543-547 to view analytics and comparison details, including expanding all or collapsing all.

Some non-limiting examples of metrics are as follows: annotations from which a surgeon can select (e.g., phases, instruments, clips/needles, surgical events, anatomy/critical structures, camera in/out, ICG in/out, surgeon swap, annotations added by the surgeon), annotations included in the surgical procedure (e.g., first appearance of an annotation/multiple annotations, last appearance of an annotation/multiple annotations, all instances), conditions for showing or calculating metrics (e.g., cases with certain case tags (e.g., calculate X for cases with grade 4 complexity)), coinciding with certain annotation time periods (e.g., calculate X for port insertion phase)), operations (e.g., sum of time periods for an annotation/multiple annotations, overlap between two annotations, time(s) between two annotations, normalize an operation with another duration, count of an annotation or sequence of annotations), multiple operations and the ordering of operations), and/or the like, including combinations and/or multiples thereof.

Some further non-limiting examples of metrics are as follows: time between first appearance of anatomy X until last appearance of phase Y, overlap between instrument X and anatomy Y during phase Z, count of times the camera went out during phase X, time between first view of anatomy X until first view of anatomy Y, count of times an instrument X went out of view, percentage of time an instrument X was in view during phase Y, count number of toggles between phase X and phase Y, and/or the like, including combinations and/or multiples thereof.

Some further non-limiting examples of metrics relating to a lap chole surgical procedure are as follows: time between start of “Dissection of Calots Triangle” phase and first view of “Cystic Duct,” time between first appearance of “Cystic Duct” and “Cystic Artery,” overlap between “monopolar shears” in view during “Dissection of Calots Triangle,” number of times the camera went out in the procedure, number of times the camera went out during “Dissection of Calots Triangle,” number of times monopolar shears went out of view during “Dissection of Calots Triangle,” number of toggles between “Dissection of Calots Triangle” and “Gallbladder dissection” phases, and/or the like, including combinations and/or multiples thereof.

According to an embodiment, the video analysis dashboard 501 can provide for linking to a procedure metrics editor. This provides for a viewer of the video analysis dashboard to create procedure metrics based on machine learning or manual annotations (e.g., overlap or time between a phase/anatomy annotation), for example, time between Calot's triangle dissection and cystic duct.

According to an embodiment, the video analysis dashboard 501 can provide for viewing videos with similar criteria (e.g., similar criteria to the current surgical procedure) within a similar case region 542. A user can select to have more or less matching criteria (see, e.g., the video analysis dashboard 516 of FIG. 5P).

It is understood that one or more embodiments described herein is capable of being implemented in conjunction with any other type of computing environment now known or later developed. For example, FIG. 6 depicts a block diagram of a processing system 600 for implementing the techniques described herein. In examples, processing system 600 has one or more central processing units (“processors” or “processing resources” or “processing devices”) 621a, 621b, 621c, etc. (collectively or generically referred to as processor(s) 621 and/or as processing device(s)). In aspects of the present disclosure, each processor 621 can include a reduced instruction set computer (RISC) microprocessor. Processors 621 are coupled to system memory (e.g., random access memory (RAM) 624) and various other components via a system bus 633. Read only memory (ROM) 622 is coupled to system bus 633 and may include a basic input/output system (BIOS), which controls certain basic functions of processing system 600.

Further depicted are an input/output (I/O) adapter 627 and a network adapter 626 coupled to system bus 633. I/O adapter 627 may be a small computer system interface (SCSI) adapter that communicates with a hard disk 623 and/or a storage device 625 or any other similar component. I/O adapter 627, hard disk 623, and storage device 625 are collectively referred to herein as mass storage 634. Operating system 640 for execution on processing system 600 may be stored in mass storage 634. The network adapter 626 interconnects system bus 633 with an outside network 636 enabling processing system 600 to communicate with other such systems.

A display 635 (e.g., a display monitor) is connected to system bus 633 by display adapter 632, which may include a graphics adapter to improve the performance of graphics intensive applications and a video controller. In one aspect of the present disclosure, adapters 626, 627, and/or 632 may be connected to one or more I/O busses that are connected to system bus 633 via an intermediate bus bridge (not shown). Suitable I/O buses for connecting peripheral devices, such as hard disk controllers, network adapters, and graphics adapters typically include common protocols, such as the Peripheral Component Interconnect (PCI). Additional input/output devices are shown as connected to system bus 633 via user interface adapter 628 and display adapter 632. A keyboard 629, mouse 630, and speaker 631 may be interconnected to system bus 633 via user interface adapter 628, which may include, for example, a Super I/O chip integrating multiple device adapters into a single integrated circuit.

In some aspects of the present disclosure, processing system 600 includes a graphics processing unit 637. Graphics processing unit 637 is a specialized electronic circuit designed to manipulate and alter memory to accelerate the creation of images in a frame buffer intended for output to a display. In general, graphics processing unit 637 is very efficient at manipulating computer graphics and image processing, and has a highly parallel structure that makes it more effective than general-purpose CPUs for algorithms where processing of large blocks of data is done in parallel.

Thus, as configured herein, processing system 600 includes processing capability in the form of processors 621, storage capability including system memory (e.g., RAM 624), and mass storage 634, input means, such as keyboard 629 and mouse 630, and output capability including speaker 631 and display 635. In some aspects of the present disclosure, a portion of system memory (e.g., RAM 624) and mass storage 634 collectively store the operating system 640 to coordinate the functions of the various components shown in processing system 600.

It is to be understood that the block diagram of FIG. 6 is not intended to indicate that the computer system 600 is to include all of the components shown in FIG. 6. Rather, the computer system 600 can include any appropriate fewer or additional components not illustrated in FIG. 6 (e.g., additional memory components, embedded controllers, modules, additional network interfaces, etc.). Further, the aspects described herein with respect to computer system 600 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language, such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium, such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be

[symbol]

The invention may be described by reference to the following numbered paragraphs:
1. A computer-implemented method comprising:
   receiving a video of a surgical procedure;
   analyzing the video of the surgical procedure to identify a feature of the surgical procedure; and
   generating a video analysis dashboard based at least in part on the feature of the surgical procedure, the video analysis dashboard comprising:
      a video region to display the video of the surgical procedure;
      a case summary region to display a case summary of the surgical procedure;
      a timeline reel region to display timelines of the surgical procedure and configured to allow selection of times within the timeline reel; and
      an analytics region to display analytics of the surgical procedure.
2. The computer-implemented method of paragraph 1, wherein the timeline reel region provides for creating a highlight reel and downloading the highlight reel.
3. The computer-implemented method of paragraph 1, wherein the video includes an augmented reality element associated with a feature of the video.
4. The computer-implemented method of paragraph 1, wherein the case summary comprises a plurality of key moments and timestamps associated with each of the plurality of key moments.
5. The computer-implemented method of paragraph 4, wherein the key moments are identified using a machine learning algorithm or a statistical analysis.
6. The computer-implemented method of paragraph 1, wherein the timeline reel region comprises at least one selected from the group consisting of an events timeline, a phases timeline, a camera timeline, a surgeons timeline, an anatomy timeline, and an instruments timeline.
7. The computer-implemented method of paragraph 6, wherein the timeline reel region comprises an add anatomy option to add an anatomy to the timeline.
8. The computer-implemented method of paragraph 6, wherein the timeline reel region comprises an add instruments option to add an instrument to the timeline.
9. The computer-implemented method of paragraph 1, wherein the analytics region comprises at least one selected from the group consisting of a metrics overview, a phase analysis, an energy instrument usage, a critical structure viability, user created metrics, and procedure specific metrics.
10. The computer-implemented method of paragraph 1, wherein data for the analytics region is based on an average, wherein the average is an average for a particular surgeon who performed the surgical procedure, an average for a group of surgeons associated with the particular surgeon, or a global average for a global population of surgeons.
11. The computer-implemented method of paragraph 1, wherein data for the analytics region is based on a statistical analysis of the video, wherein the statistical analysis includes determining an average, a standard deviation and outlier detection.
12. The computer-implemented method of paragraph 1, wherein the video analysis dashboard further comprises a similar case region to display one or more similar cases relative to the surgical procedure.
13. The computer-implemented method of paragraph 1, wherein the video is captured by a camera.
14. The computer-implemented method of paragraph 1, wherein the video is captured by an ultrasound device.
15. A system, comprising:
   a processor configured to receive a video of a surgical procedure from non-transitory memory;
   wherein the processor is configured to analyze the video of the surgical procedure to identify a feature of the surgical procedure and generate a video analysis dashboard based on the feature of the surgical procedure, the video analysis dashboard comprising:
      a video region to display the video of the surgical procedure;
      a case summary region to display a case summary of the surgical procedure;
      a timeline reel region to display highlights of the surgical procedure; and
      an analytics region to display analytics of the surgical procedure; and
   wherein a display provides a visual display of the output from the video analysis dashboard.
16. The system of paragraph 15, wherein the timeline reel region provides for creating a highlight reel and downloading the highlight reel.
17. The system of paragraph 15, wherein the video includes an augmented reality element associated with a feature of the video.
18. The system of paragraph 15, wherein the case summary comprises a plurality of key moments and timestamps associated with each of the plurality of key moments and wherein the key moments are identified using a machine learning algorithm or a statistical analysis.
19. The system of paragraph 15, wherein the timeline reel region comprises at least one selected from the group consisting of an events timeline, a phases timeline, a camera timeline, a surgeons timeline, an anatomy timeline, and an instruments timeline.
20. The system of paragraph 19, wherein the timeline reel region comprises an add anatomy option to add one or more anatomy or instrument timelines.
21. The system of paragraph 15, wherein the analytics region comprises at least one selected from the group consisting of a metrics overview, a phase analysis, an energy instrument usage, a critical structure viability, user created metrics, and user or pre-defined procedure specific metrics.
22. The system of paragraph 15, wherein data for the analytics region is based on an average in general or for cases with the same case tags, wherein the average is an average for a particular surgeon who performed the surgical procedure, an average for a group of surgeons associated with the particular surgeon, or a global average for a global population of surgeons.
23. The system of paragraph 15, wherein data for the analytics region is based on a statistical analysis of the video, wherein the statistical analysis includes determining an average, a standard deviation and outlier detection.
24. The system of paragraph 15, wherein the video analysis dashboard further comprises a similar case region to display one or more similar cases relative to the surgical procedure.
25. The system of paragraph 15, wherein the video is captured by a camera or ultrasound device.

What is claimed is:

1. A computer-implemented method comprising:
receiving a video of a surgical procedure;
analyzing the video of the surgical procedure to identify a feature of the surgical procedure; and
generating a video analysis dashboard based at least in part on the feature of the surgical procedure, the video analysis dashboard comprising:
a video region to display the video of the surgical procedure;
a case summary region to display a case summary of the surgical procedure;
a timeline reel region to display timelines of the surgical procedure and configured to allow selection of times within the timeline reel; and
an analytics region to display analytics of the surgical procedure.

2. The computer-implemented method of claim 1, wherein the timeline reel region provides for creating a highlight reel and downloading the highlight reel.

3. The computer-implemented method of claim 1, wherein the video includes an augmented reality element associated with a feature of the video.

4. The computer-implemented method of claim 1, wherein the case summary comprises a plurality of key moments and timestamps associated with each of the plurality of key moments, and the key moments are identified using a machine learning algorithm or a statistical analysis.

5. The computer-implemented method of claim 1, wherein the timeline reel region comprises at least one selected from the group consisting of an events timeline, a phases timeline, a camera timeline, a surgeons timeline, an anatomy timeline, and an instruments timeline, and the timeline reel region comprises an add anatomy option to add an anatomy to the timeline and/or an add instruments option to add an instrument to the timeline.

6. The computer-implemented method of claim 1, wherein the analytics region comprises at least one selected from the group consisting of a metrics overview, a phase analysis, an energy instrument usage, a critical structure viability, user created metrics, and procedure specific metrics.

7. The computer-implemented method of claim 1, wherein data for the analytics region is based on an average, wherein the average is an average for a particular surgeon who performed the surgical procedure, an average for a group of surgeons associated with the particular surgeon, or a global average for a global population of surgeons.

8. The computer-implemented method of claim 1, wherein data for the analytics region is based on a statistical analysis of the video, and the statistical analysis includes determining an average, a standard deviation and outlier detection.

9. The computer-implemented method of claim 1, wherein the video analysis dashboard further comprises a similar case region to display one or more similar cases relative to the surgical procedure.

10. The computer-implemented method of claim 1, wherein the video is captured by a camera or by an ultrasound device.

11. A data carrier or memory storing a set of instructions for a processor which when executed carries out a method comprising:
analyzing a video of a surgical procedure to identify a feature of the surgical procedure using a machine learning algorithm; and
generating a video analysis dashboard based at least in part on the feature of the surgical procedure, the video analysis dashboard comprising:
a video region to display the video of the surgical procedure;
a case summary region to display a case summary of the surgical procedure;
a timeline reel region to display timelines of the surgical procedure and configured to allow selection of times within the timeline reel; and
analytics region to display analytics of the surgical procedure.

12. A system, comprising:
a processor configured to receive a video of a surgical procedure from non-transitory memory;
wherein the processor is configured to analyze the video of the surgical procedure to identify a feature of the surgical procedure and generate a video analysis dashboard based on the feature of the surgical procedure, the video analysis dashboard comprising:
a video region to display the video of the surgical procedure;
a case summary region to display a case summary of the surgical procedure;
a timeline reel region to display highlights of the surgical procedure; and
an analytics region to display analytics of the surgical procedure; and
wherein a display provides a visual display of the output from the video analysis dashboard.

13. The system of claim 12, wherein the timeline reel region provides for creating a highlight reel and downloading the highlight reel.

14. The system of claim 12, wherein the video includes an augmented reality element associated with a feature of the video.

15. The system of claim 12, wherein the case summary comprises a plurality of key moments and timestamps associated with each of the plurality of key moments and wherein the key moments are identified using a machine learning algorithm or a statistical analysis.

16. The system of claim 12, wherein the timeline reel region comprises at least one selected from the group consisting of an events timeline, a phases timeline, a camera timeline, a surgeons timeline, an anatomy timeline, and an instruments timeline, and the timeline reel region comprises an add anatomy option to add one or more anatomy or instrument timelines.

17. The system of claim 12, wherein the analytics region comprises at least one selected from the group consisting of a metrics overview, a phase analysis, an energy instrument usage, a critical structure viability, user created metrics, and user or pre-defined procedure specific metrics.

18. The system of claim 12, wherein data for the analytics region is based on an average in general or for cases with the same case tags, wherein the average is an average for a particular surgeon who performed the surgical procedure, an average for a group of surgeons associated with the particular surgeon, or a global average for a global population of surgeons.

19. The system of claim 12, wherein data for the analytics region is based on a statistical analysis of the video, wherein the statistical analysis includes determining an average, a standard deviation and outlier detection, the video analysis dashboard further comprises a similar case region to display one or more similar cases relative to the surgical procedure, and the video is captured by a camera or ultrasound device.
